SEQUENCE LISTING (4 sequences)

SEQ ID NO: 1 - DNA, 20 bp, Mus musculus
cctatgctggggacaacatc

SEQ ID NO: 2 - DNA, 20 bp, Mus musculus
tgcacctcatcatcttccag

SEQ ID NO: 3 - DNA, 20 bp, Mus musculus
ggtgaaggtcggtgtgaacg

SEQ ID NO: 4 - DNA, 20 bp, Mus musculus
ctcgctcctggaagatggtg

(19) United States
(12) Patent Application Publication
(10) Pub. No.: US 2016/0279181 A1
(43) Pub. Date: Sep. 29, 2016

(54) INTESTINAL BARRIER FUNCTION ENHANCER CONTAINING LACTIC ACID BACTERIA

(71) Applicant: SUNTORY HOLDINGS LIMITED, Osaka-shi, Osaka (JP)

(72) Inventor: Hiroyuki HOSHIKO, Osaka (JP)

(73) Assignees: SUNTORY HOLDINGS LIMITED, Osaka-shi, Osaka (JP); SUNTORY HOLDINGS LIMITED, Osaka-shi, Osaka (JP)

(21) Appl. No.: 15/032,733

(22) PCT Filed: Nov. 7, 2014
(86) PCT No.: PCT/JP2014/079598

§ 371 Date: Apr. 28, 2016

(30) Foreign Application Priority Data
Nov. 7, 2013 (JP) 2013-231140

(51) Int. Cl. A61K 35/747; A23L 2/52

(52) U.S. Cl. CPC A61K 35/747; A23L 1/3014; A23L 2/52; A23Y 2220/00

(57) ABSTRACT

Provided are an intestinal barrier function enhancer capable of enhancing intestinal barrier function and thereby capable of preventing or treating metabolic syndrome, and a drug and a food or drink containing the same. An intestinal barrier function enhancer containing cells or a treated cell product of Lactobacillus crustorum. This intestinal barrier function enhancer functions effectively to enhance intestinal barrier function and to prevent or treat conditions or diseases caused by impaired intestinal barrier function such as metabolic syndrome and the like.

### TECHNICAL FIELD

The present invention relates to foods and beverages and an intestinal barrier function enhancer, which comprise a lactic acid bacterium, as well as pharmaceutical compositions, foods and beverages which comprise said intestinal barrier function enhancer. More particularly, this invention relates to foods and beverages and an intestinal barrier function enhancer, which comprise a Lactobacillus crustorum bacterium, as well as pharmaceutical compositions, foods and beverages which comprise said intestinal barrier function enhancer.

### BACKGROUND ART

Lifestyle-related diseases such as diabetes, hypertension, and hyperlipidemia are closely related to visceral fat obesity. Conditions in which not only visceral fat obesity but also at least two of hyperglycemia, hypertension and hyperlipidemia are developed concurrently are called metabolic syndrome (or visceral fat syndrome). Particularly in developed countries where citizens are well fed, the number of patients with metabolic syndrome is on an increasing trend and there is a concern about an increase in medical expenses; thus, a countermeasure against metabolic syndrome is urgently needed.

It was reported that the common pathological condition underlying metabolic syndrome is mild chronic inflammation occurring in the body (Non-patent Literatures 2 and 3). There are known inflammation-triggering substances such as saturated fatty acids, alcohol, and periodontal bacteria.

In recent years, it was also reported that intestinal bacteria and intestinal environment are related to obesity accompanied with chronic inflammation (Non-patent Literature 4). The human intestine is inhabited by over 100 different species of over 100 trillion intestinal bacteria per individual, which form the intestinal flora (gut microbiota). Some intestinal bacteria can trigger an inflammatory reaction in the body. For example, it was found that lipopolysaccharide (LPS), a component of the cell walls of gram-negative intestinal bacteria, triggers the release of inflammatory cytokines in mice and humans (Non-patent Literature 4).

Underneath intestinal epithelial cells, there exist a great number of immune cells such as macrophages, dendritic cells, T-cells and B-cells. Under normal conditions, intestinal epithelial cells are tightly joined to each other by tight junctions, so that high molecular weight substances are strictly controlled so as not to pass through intercellular spaces. Also, intestinal epithelial cells contain transporters for removing hydrophobic foreign substances from the cells. The aforementioned tight junction structure and transporters are mainly responsible for intestinal barrier function which prevents the entry of foreign substances. However, when the intestinal barrier is damaged by saturated fatty acids, alcohol, or any other factor, leading to an increase in intestinal permeability, high molecular weight substances such as intestinal bacteria and their bacterial components, which are present in the intestine, penetrate through intercellular spaces deeper into the body and stimulate immune cells to release inflammatory cytokines, thereby inducing inflammation.

There is a demand for a means that is capable of enhancing intestinal barrier function to prevent chronic inflammation, thereby enabling treatment or prevention of metabolic syndrome.

Lactic acid-producing bacteria, i.e., lactic acid bacteria, are known to play an important role in maintaining the balance of the intestinal flora and keeping a healthy intestinal environment. Various studies have been made on the influence of lactic acid bacteria on intestinal barrier function; for example, there was a report that Lactobacillus bacteria which are capable of adhering to intestinal cells can enhance the intestinal permeability of minerals such as calcium (Patent Literature 1). It was also reported that Lactobacillus rhamnosus OLL2838, Lactobacillus delbrueckii subsp. bulgaricus MEP190901, Lactobacillus gasseri MEP190903, and Lactobacillus gasseri MEP190903, as well as their bacterial component, lipoteichoic acid, have a capability of suppressing an increase in intestinal permeability (Patent Literature 2).

### CITATION LIST

### Patent Literatures

Patent Literature 1: Japanese Patent Domestic Publication No. JP 2002-507997

Patent Literature 2: Japanese Patent Application Publication No. JP 2008-212006

### Non-Patent Literatures

Non-patent Literature 1: Japan National Health and Nutrition Survey 2011

Non-patent Literature 2: FEBS Lett., 582, 97-105 (2008)

Non-patent Literature 3: J. Clin. Invest., 116, 1793-1801 (2006)

Non-patent Literature 4: Journal of Intestinal Microbiology, 24, 193-201 (2010)

### SUMMARY OF INVENTION

### Technical Problem

The present invention has as its object to provide an intestinal barrier function enhancer that is capable of enhancing intestinal barrier function to thereby enable prevention or treatment of metabolic syndrome, as well as pharmaceutical products, foods and beverages comprising said intestinal barrier function enhancer.

### Solution to Problem

The present inventors found that particular Lactobacillus crustorum bacteria increase a transepithelial resistance value, a measure of intestinal barrier function in cultured human intestinal epithelial cells. The inventors also found that those bacteria are capable of inducing the expression of tight junction-related genes in cultured human intestinal epithelial cells and in the murine small intestine epithelium. The inventors further found that those bacteria, when administered to mice fed a high fat diet, exhibit inhibitory activities against glucose metabolism disorders. Thus, the inventors have completed the present invention.

More specifically, the present invention includes but is not limited to the following.

[1] A food or beverage comprising a Lactobacillus crustorum bacterium or a treated product of said bacterium.
[2] The food or beverage as set forth in [1], wherein the Lactobacillus crustorum bacterium is Lactobacillus crustorum SAM2695 strain, Lactobacillus crustorum SAM2697 strain, Lactobacillus crustorum SAM2698 strain, Lactobacillus crustorum JCM 15951^{T} strain, or a combination of two or more thereof.
[3] The food or beverage as set forth in [1] or [2], wherein the food or beverage comprises a viable Lactobacillus crustorum bacterium.
[4] An intestinal barrier function enhancer comprising a Lactobacillus crustorum bacterium or a treated product of said bacterium.
[5] The intestinal barrier function enhancer as set forth in [4], wherein the Lactobacillus crustorum bacterium is Lactobacillus crustorum SAM2695 strain, Lactobacillus crustorum SAM2697 strain, Lactobacillus crustorum SAM2698 strain, Lactobacillus crustorum JCM 15951^{T} strain, or a combination of two or more thereof.
[6] The intestinal barrier function enhancer as set forth in [4] or [5], wherein the food or beverage comprises a viable Lactobacillus crustorum bacterium.
[7] A pharmaceutical composition for prevention or treatment of at least one condition or disease selected from the group consisting of diabetes, hypertension, hyperlipidemia and obesity, the pharmaceutical composition comprising the intestinal barrier function enhancer as set forth in any of [4] to [6].
[8] A food or beverage comprising the intestinal barrier function enhancer as set forth in any of [4] to [6].
[9] The food or beverage as set forth in [8], wherein the food or beverage is intended for use in prevention or treatment of at least one condition or disease selected from the group consisting of diabetes, hypertension, hyperlipidemia and obesity.
[10] The pharmaceutical composition as set forth in [7], or the food or beverage as set forth in [9], wherein the condition or disease is diabetes.
[11] The pharmaceutical composition as set forth in [7], or the food or beverage as set forth in [9], wherein the condition or disease is a combination of obesity with at least one disease selected from the group consisting of diabetes, hypertension and hyperlipidemia.
[12] The pharmaceutical composition, or food or beverage as set forth in any of [7] to [11], wherein the diabetes is Type 2 diabetes.
[13] A method for preparing a pharmaceutical composition or a food or beverage, the method comprising the step of incorporating the intestinal barrier function enhancer as set forth in any of [4] to [6].
[14] The method as set forth in [13], wherein the pharmaceutical composition or the food or beverage is intended for use in prevention or treatment of at least one condition or disease selected from the group consisting of diabetes, hypertension, hyperlipidemia and obesity.
[15] The method as set forth in [14], wherein the condition or disease is diabetes.
[16] The method as set forth in [14], wherein the condition or disease is a combination of obesity with at least one disease selected from diabetes, hypertension and hyperlipidemia.
[17] The method as set forth in any of [13] to [16], wherein the diabetes is Type 2 diabetes.

### Advantageous Effects of Invention

The intestinal barrier function enhancer of the present invention is capable of enhancing intestinal barrier function due to its comprising a Lactobacillus crustorum bacterium or a treated product of said bacterium. Also, the inventive intestinal barrier function enhancer can be effectively used for pharmaceutical compositions, foods or beverages which are intended for use in prevention or treatment of various conditions or diseases attributable to intestinal barrier dysfunction, including diabetes, hypertension, hyperlipidemia, obesity, and metabolic syndrome.

### BRIEF DESCRIPTION OF DRAWINGS

FIG. 1 shows the results of the evaluation of enhancement of intestinal barrier function by Lactobacillus crustorum bacteria using Caco2 human intestinal epithelial cells.

FIG. 2 shows the results of the microarray analysis in the Caco2 intestinal epithelial cells cocultured with a Lactobacillus crustorum bacterium.

FIG. 3 shows the change in the expression of Claudin 1 (CLDN 1) gene in small-intestinal epithelial tissue of the mice treated with a Lactobacillus crustorum bacterium.

FIG. 4 shows the inhibitory activities of Lactobacillus crustorum strains against glucose metabolism disorders in high-fat diet-loaded model mice.

FIG. 5 shows the inhibitory activities of Lactobacillus crustorum strains against glucose metabolism disorders in high-fat diet-loaded model mice.

### DESCRIPTION OF EMBODIMENTS

<Lactobacillus crustorum>

Lactobacillus crustorum, a species of lactic acid bacteria belonging to the Lactobacillus genus, was proposed to be classified as a novel Lactobacillus species isolated from wheat sourdoughs (International Journal of Systematic and Evolutionary Microbiology (2007), 57, 1461-1467). Sourdough refers to a type of bread obtained by preparing a dough with a mixture of wheat or rye flour and water and fermenting the dough using different types of microorganisms including lactic acid bacteria and yeast.

Lactobacillus crustorum has the following mycological properties.

### Morphological Properties

(1) Bacillus with a length of 2-15 μm and a width of 0.5-1 μm
(2) Non-motile
(3) Non-sporulating

### Cultural Properties

(1) Grows well in a liquid or solid MRS medium under anaerobic or aerobic conditions.
(2) Grows at a temperature of 15° C. in the presence of 5, 6 or 7% salt.
(3) Grows at a temperature of 45° C. in the presence of 5, 6 or 7% salt.

### Physiological Properties

(1) Gram-staining: Negative
(2) Tested with catalase
(3) Fermentation: Homolactic
(4) Ammonium production (from arginine): No
(5) Gas production (glucose or gluconate): No
(6) Acid production (for LMG 23699, LMG 23701, LMG 23702, and R-30103 strains):

Galactose, glucose, fructose, mannose, N-acetylglucosamine, esculin and salicin: Acid produced.

Glycerol, erythritol, D-arabinose, L-arabinose, ribose, D-xylose, L-xylose, adonitol, β-methyl-D-xyloside, sorbose, dulcitol, inositol, mannitol, sorbitol, α-methyl-D-mannoside, α-methyl-D-glucoside, melibiose, sucrose, inulin, melezitose, raffinose, starch, glycogen, xylitol, D-turanose, D-lyxose, D-fucose, L-fucose, D-arabitol, L-arabitol, gluconate, 2-ketogluconate, and 5-ketogluconate: No acid produced.

In the present invention, any bacterial strain can be used as long as it is a bacterium that has the aforementioned mycological properties and which is classified as belonging to the species Lactobacillus crustorum. For example, the SAM2695, SAM2697 and SAM2698 strains isolated by Tokyo University of Agriculture can be mentioned as examples. The SAM2695, SAM2697 and SAM2698 strains were deposited to NITE Patent Microorganisms Depositary (NPMD), National Institute of Technology and Evaluation (2-5-8 Kazusakamatari, Kisarazu-shi, Chiba 292-0818, Japan) under the accession Nos. NITE BP-01721, NITE BP-01722 and NITE BP-01723, respectively, as of Oct. 17, 2013. Lactobacillus crustorum LMG 23699^{T}, a type culture strain available from Belgian Co-ordinated Collections of Micro-organisms, can also be used. Lactobacillus crustorum LMG 23699^{T} is also available from Japan Collection of Microorganisms, RIKEN BioResource Center under the accession No. JCM 15951^{T}. In this invention, any one of such strains as mentioned above, or a combination of two or more thereof, can be used. For example, the intestinal barrier function enhancer of this invention may comprise one strain of the species Lactobacillus crustorum, or two or more strains of said species.

The Lactobacillus crustorum bacterium to be used for the present invention can be cultured using any liquid or solid medium capable of culturing lactic acid bacteria. Examples of the culture medium include a MRS medium (MRS broth) and a MRS agar medium (Sharpe M., et al., “Identification of the Lactic Acid Bacteria”, Identification Method for Microbiologists Part A, London and New York, Academic Press, p. 65-79, 1966). As for the MRS medium and the MRS agar medium, any such medium may be prepared according to a conventional procedure, or a commercially available one from, for example, Kanto Chemical Co., Inc. may be used. Also, milk from a mammal such as cattle, horse or sheep can be used as a medium. The culture temperature of Lactobacillus crustorum can be set to be in the range of 15 to 45° C., as appropriate, with 30° C. being preferred. As for the culture conditions of Lactobacillus crustorum, both anaerobic and aerobic cultures can be used.

<Intestinal Barrier Function Enhancer>

The intestinal barrier function enhancer of the present invention, which comprises a Lactobacillus crustorum bacterium or a treated product of said bacterium, has a capability of enhancing intestinal barrier function.

As referred to in the present invention, the “intestinal barrier function” refers to a function of preventing the entry of a substance from the outside of intestinal epithelial cells (i.e., the inside of the intestine) deeper into the body. A state in which a substance is stimulated to enter from the outside of intestinal epithelial cells deeper into the body as compared to normal state is described as a state of increased permeability in intestinal epithelial cells. Enhancement of intestinal barrier function means both suppression of an increase in permeability in intestinal epithelial cells, and reduction of permeability in intestinal epithelial cells. For example, enhancement of intestinal barrier function is achieved by normalizing or strengthening a tight junction structure in which intestinal epithelial cells are adhered to each other, or by normalizing or strengthening the function of transporters present in intestinal epithelial cells, which is to remove a foreign matter from the cells.

The effects of enhancement of intestinal barrier function are represented, for example, by a decrease in the amount of a particular substance migrating from the inside of the intestine through intestinal epithelial cells and deeper in the body, or by an increase in the transepithelial electric resistance (TER) value of intestinal epithelial cells. One skilled in the art can select a specific method for evaluating the effects of enhancement of intestinal barrier function depending on the purpose; for example, there can be advantageously used a method for determining a TER value in human intestinal epithelial cells (Caco2 cells), as described later in the Examples section. To be specific, if cultured Caco2 monolayer cells to which a test substance is added show an increase in TER as compared to control cells, said test substance can be determined to be effective in enhancement of intestinal barrier function.

The intestinal barrier function enhancer of the present invention comprises a Lactobacillus crustorum bacterium or a treated product of said bacterium. For the purpose of the present specification, the “bacterium/bacteria” is used as a term that means both viable and non-viable bacteria. The bacterium may be the one purified by removal of a culture supernatant or may be the one containing impurities such as a solid or liquid medium. Also, the bacterium to be incorporated in the inventive intestinal barrier function enhancer can be in a wet state or in a dry state. In the case of using the bacterium in a wet state, for example, a culture comprising a Lactobacillus crustorum bacterium and a solid or liquid medium can be incorporated in the inventive intestinal barrier function enhancer, as it is, or in the form of a concentrate obtained by concentrating the culture. In the case of using the bacterium in a dry state, a Lactobacillus crustorum bacterium dried by spray drying, freeze drying, vacuum drying or other drying method can be incorporated in the intestinal barrier function enhancer, as it is, or in a powdered or granulated form. Non-viable bacteria are obtained by sterilizing a culture of a Lactobacillus crustorum bacterium, a concentrate of such a culture, a purified bacterium, or a dried bacterium by heat treatment, radiation treatment, or any other treatment. Also, the inventive intestinal barrier function enhancer can comprise a treated product of a Lactobacillus crustorum bacterium. The “treated product of a bacterium” refers to a crushed or pulverized product of bacterium typically obtained by ultrasonic or physical crushing or pulverization, a bacterial lysate obtained by enzymatic treatment with a cell wall lysing enzyme or the like, an immobilized bacterium obtained by immobilizing a bacterium, for example, on a solid carrier or in a microcapsule, or a cultured product remaining after removal of a bacterium, such as a supernatant of a bacterial culture solution.

In the present invention, a Lactobacillus crustorum bacterium or a treated product of said bacterium can be directly used as an intestinal barrier function enhancer. Also, a Lactobacillus crustorum bacterium or a treated product of said bacterium, with a pharmacologically acceptable carrier or excipient being added thereto, can be used as an intestinal barrier function enhancer. Exemplary carriers include, but are not limited to, culture medium, water, physiological saline, ethanol, propylene glycol, and glycerol. Exemplary excipients include, but are not limited to, glucose, sucrose, lactose, dextrin, cyclodextrin, xanthan gum, guar gum, gum arabic, gum tragant, gellan gum, locust bean gum, carrageenan, pectin, and agar. Other components commonly used in formulating pharmaceutical preparations, such as emulsifier, isotonizer, buffer, solubilizer, antiseptic, stabilizer, and antioxidant, may be added as appropriate.

The amount of a Lactobacillus crustorum bacterium or a treated product of said bacterium to be incorporated as an active component of the inventive intestinal barrier function enhancer can be determined as appropriate in consideration of effects. For example, in the case of using a Lactobacillus crustorum bacterium as an active component, said bacterium is present in the intestinal barrier function enhancer in an amount of preferably from 0.00001 to 99.9% by weight, more preferably from 0.0005 to 50% by weight, yet more preferably from 0.0001 to 10% by weight, as calculated on the basis of the dry weight of said bacterium. Alternatively, from the viewpoint of the number of bacteria, the bacterium is present in an amount ranging preferably from 1.0×10² to 1.0×10¹²/g, more preferably from 1.0×10⁶ to 1.0×10¹²/g. In the case of using a treated product of a Lactobacillus crustorum bacterium as an active component, said treated product is present in the intestinal barrier function enhancer in an amount of preferably from 0.00001 to 99.9% by weight, more preferably from 0.0005 to 50% by weight, yet more preferably from 0.0001 to 10% by weight, as calculated on a solids weight basis. In the case of using a combination of a Lactobacillus crustorum bacterium with a treated product of said bacterium as an active component, said combination is present in the intestinal barrier function enhancer in an amount of preferably from 0.00001 to 99.9% by weight, more preferably from 0.0005 to 50% by weight, yet more preferably from 0.0001 to 10% by weight, as calculated on the basis of the total of the dry weight of said bacterium and the solids weight of said treated product.

<Pharmaceutical Composition Comprising an Intestinal Barrier Function Enhancer>

The present invention also provides a pharmaceutical composition comprising the aforementioned intestinal barrier function enhancer.

The inventive pharmaceutical composition, because of comprising an intestinal barrier function enhancer, is effective for prevention or treatment of conditions or diseases related to intestinal barrier dysfunction. Such conditions or diseases related to intestinal barrier dysfunction include, but are not limited to, obesity, diabetes, hypertension, hyperlipidemia, inflammatory bowel diseases, and food allergy. As referred to herein, the state of obesity refers to a state in which the proportion of body weight relative to body height is increased due to an increase in the number of fat cells or accumulation of excessive fat droplets in fat cells. The state of obesity also covers a state not reaching the disease adiposis (a pathological condition in which health problems attributable or related to obesity coexist or are predicted to coexist). A state in which obesity is accompanied with at least one disease selected from the group consisting of diabetes, hypertension and hyperlipidemia can be regarded as a stage prior to metabolic syndrome. A state in which obesity is accompanied with two or more diseases selected from the group consisting of diabetes, hypertension and hyperlipidemia is called “metabolic syndrome.”

As referred to herein, the “prevention of a condition or disease” refers to enhancement of the resistance of a subject to the condition or disease, or prevention of the onset of the condition or disease. As referred to herein, the “treatment of a condition or disease” refers to recovery of a subject from the condition or disease, improvement of the severity of the condition or disease, or prevention of the progress of the condition or disease.

The pharmaceutical composition of the present invention can be formulated into a pharmaceutical preparation with a pharmacologically acceptable carrier, excipient or the like according to a common procedure depending on the purpose. Exemplary carriers include, but are not limited to, culture medium, water, physiological saline, ethanol, propylene glycol, and glycerol. Exemplary excipients include, but are not limited to, glucose, sucrose, lactose, dextrin, cyclodextrin, xanthan gum, guar gum, gum arabic, gum tragant, gellan gum, locust bean gum, carrageenan, pectin, and agar. Other components commonly used in formulating pharmaceutical preparations, such as emulsifier, isotonizer, buffer, solubilizer, antiseptic, stabilizer, and antioxidant, may be added as appropriate.

The pharmaceutical composition of the present invention may be optionally mixed with any other additives, including minerals; vitamins such as vitamin E, vitamin C and vitamin A; nutrients; flavorants; and pigments, as long as said additives do not impair the effects of a Lactobacillus crustorum bacterium or a treated product of said bacterium in enhancing intestinal barrier function, or in other words, as long as mixing of said bacterium or treated product with said additives does not produce an unfavorable interaction. As such additives, any additives commonly used in pharmaceutical products can be used. Further, the inventive pharmaceutical composition may be mixed with any one or more other physiologically active components, as long as said physiologically active components do not impair the effects of a Lactobacillus crustorum bacterium or a treated product of said bacterium in enhancing intestinal barrier function.

The pharmaceutical composition of the present invention can also be used in combination with other pharmaceutical agent. In the case of using the inventive pharmaceutical composition in combination with other pharmaceutical agent, the inventive pharmaceutical composition and such other pharmaceutical agent may be used in the form of one composition comprising both of them in combination, or may be used in a combined treatment therapy in which the inventive pharmaceutical composition and such other pharmaceutical agent are each used in a form contained in a separate formulation. In the process of the combined therapy, administration of the inventive pharmaceutical composition can be done before, during or after administration of such other pharmaceutical agent.

Examples of said other pharmaceutical agent include: anti-obesity agents such as mazindol and orlistat; anti-diabetic agents such as tolbutamide, nateglinide, acarbose, metformin, pioglitazone, sitagliptin and epalrestat; diuretics such as chlorthalidone, hydrochlorothiazide, indapamide, furosemide, torasemide and spironolactone; calcium antagonists such as nifedipine, amlodipine and diltiazem; angiotensin converting enzyme inhibitors such as imidapril and benazepril; angiotensin receptor antagonists such as losartan and valsartan; direct renin inhibitors such as aliskiren; α-receptor blockers such as phentolamine and prazosin; β-receptor blockers such as atenolol and celiprolol; α₁β blockers such as carvedilol; α₂ receptor agonists such as clonidine; statin hypolipidemic agents such as mevastatin, pravastatin and atorvastatin; fibrate hypolipidemic agents such as bezafibrate and fenofibrate; resins (bile acid binders) such as colestimide; and other hypolipidemic agents such as probucol and ezetimibe.

The form of the inventive pharmaceutical composition is not particularly limited; and the pharmaceutical composition can be prepared in any form: for example, solid form such as powder, granule, or tablet; liquid form such as solution, emulsion, or dispersion; or semi-solid form such as paste. Specific examples of the dosage form of the pharmaceutical composition include powder, granule, fine granule, tablet, pill, troche, capsule (including soft and hard capsules), chewable tablet, and solution.

In one mode, the pharmaceutical composition of the present invention comprises a Lactobacillus crustorum bacterium in an amount of preferably from 0.00001 to 99.9% by weight, more preferably from 0.0001 to 10% by weight, as calculated on the basis of the dry weight of said bacterium. In another mode, the inventive pharmaceutical composition comprises a treated product of a Lactobacillus crustorum bacterium in an amount of preferably from 0.00001 to 99.9% by weight, more preferably from 0.0001 to 10% by weight, as calculated on a solids weight basis. In yet another mode, the inventive pharmaceutical composition comprises a Lactobacillus crustorum bacterium and a treated product of said bacterium in an amount of preferably from 0.00001 to 99.9% by weight, more preferably from 0.0001 to 10% by weight, as calculated on the basis of the total of the dry weight of said bacterium and the solids weight of said treated product.

The dose and administration form of the inventive pharmaceutical can be selected as appropriate depending on the subject to be treated, the pathological condition and its progress, and other requirements. For example, in the case of orally administering a Lactobacillus crustorum bacterium to e.g., a human (adult) subject for the purpose of obtaining the effects of enhancement of intestinal barrier function, it is advisable to orally ingest the pharmaceutical composition all at once or in about two or three divided doses daily such that the daily intake of said bacterium is approximately in the range of from 0.01 to 1000 mg, preferably from 0.1 to 200 mg, more preferably from 0.3 to 2 mg. Alternatively, from the viewpoint of the number of bacteria, it is advisable to orally ingest the pharmaceutical composition all at once or in a few divided doses daily such that the daily intake of said bacterium is not less than 1.0×10⁶ /kg body weight, preferably not less than 1.0×10⁷/kg body weight, more preferably not less than 1.0×10⁸/kg body weight. In the case of orally administering a treated product of a Lactobacillus crustorum bacterium to e.g., a human (adult) subject for the purpose of obtaining the effects of enhancement of intestinal barrier function, it is advisable to sequentially administer the pharmaceutical composition all at once or in about two or three divided doses daily such that the daily intake of said treated product is approximately in the range of from 0.01 to 1000 mg, preferably from 0.1 to 200 mg, more preferably from 0.3 to 2 mg, as calculated on a solids weight basis. In the case of orally administering a combination of a Lactobacillus crustorum bacterium and a treated product of said bacterium to e.g., a human (adult) subject for the purpose of obtaining the effects of enhancement of intestinal barrier function, it is advisable to sequentially administer the pharmaceutical composition all at once or in about two or three divided doses daily such that the total daily intake of said bacterium and said treated product is approximately in the range of from 0.01 to 1000 mg, preferably from 0.1 to 200 mg, more preferably from 0.3 to 2 mg, as calculated on the basis of the total of the dry weight of said bacterium and the solids weight of said treated product.

<Foods and Beverages>

The present invention also provides foods and beverages comprising the aforementioned intestinal barrier function enhancer.

Since the foods and beverages of the present invention comprise the intestinal barrier function enhancer, the foods and beverages are effective for prevention and treatment of various conditions or diseases related to intestinal barrier dysfunction, including obesity, diabetes, hypertension, hyperlipidemia, inflammatory bowel diseases, and food allergy.

The inventive foods and beverages can be of any form of, for example: beverages such as juice, milk, milk beverage, soft drink, tea beverage, and drinkable preparation; liquid foods such as soup and stew; paste foods such as jam and yogurt; semi-solid foods such as jelly and gummi candy; solid foods such as bread, cake, candy, cookie and gum; oil and fat-containing foods such as dressing and mayonnaise; and pharmaceutical preparations such as capsule, tablet and troche.

Further examples of the foods and beverages of the present invention include, but are not limited to, nutritional supplementary foods, health foods, functional foods, foods for infants, modified milk for infants, modified milk for premature infants, and foods and beverages for elderly persons.

The “nutritional supplementary foods” refers to foods in which a particular nutrient(s) is(are) enriched. The “health foods” refers to foods that are healthy or beneficial for health, and includes supplements, natural foods, diet foods, etc. The “functional foods” refers to foods for replenishing a nutrient(s) that has(have) a function of regulating the body, and is synonymous with foods for specified health use. The “foods for infants” refers to foods for feeding children up to about 6 years of age. The “foods for elderly persons” refers to foods that are treated so as to be easier to digest and absorb than untreated foods. The “modified milk for infants” refers to milk that is modified for feeding children up to about 1 year of age. The “modified milk for premature infants” refers to milk that is modified for feeding premature infants until about half a year after birth.

The form of the foods and beverages of the present invention is not particularly limited; and the foods and beverages can be prepared in any form: for example, solid form such as powder, granule, or tablet; liquid form such as solution, emulsion, or dispersion; or semi-solid form such as paste. Specific examples of the dosage form of the foods and beverages include powder, granule, fine granule, tablet, pill, troche, capsule (including soft and hard capsules), chewable tablet, and solution.

The foods and beverages of the present invention may be optionally mixed with any other additives, including minerals; vitamins such as vitamin E, vitamin C and vitamin A; nutrients; flavorants; and pigments, as long as said additives do not impair the effects of a Lactobacillus crustorum bacterium or a treated product of said bacterium in enhancing intestinal barrier function, or in other words, as long as mixing of said bacterium or treated product with said additives does not produce an unfavorable interaction. As such additives, any additives commonly used in foods and beverages can be used.

Also, the foods and beverages of the present invention may be mixed with any one or more other physiologically active components, as long as said physiologically active components do not impair the effects of a Lactobacillus crustorum bacterium or a treated product of said bacterium in enhancing intestinal barrier function.

In one mode, the foods and beverages of the present invention comprise a Lactobacillus crustorum bacterium in an amount of preferably from 0.00001 to 99.9% by weight, more preferably from 0.0001 to 10% by weight, as calculated on the basis of the dry weight of said bacterium. In another mode, the inventive foods and beverages comprise a treated product of a Lactobacillus crustorum bacterium in an amount of preferably from 0.00001 to 99.9% by weight, more preferably from 0.0001 to 10% by weight, as calculated on the basis of the solids weight of said treated product. In yet another mode, the inventive foods and beverages comprise a Lactobacillus crustorum bacterium and a treated product of said bacterium in an amount of preferably from 0.00001 to 99.9% by weight, more preferably from 0.0001 to 10% by weight, as calculated on the basis of the total of the dry weight of said bacterium and the solids weight of said treated product.

<Method for Preparing a Pharmaceutical Composition or a Food or Beverage>

The intestinal barrier function enhancer for clock genes according to the present invention, because of comprising a Lactobacillus crustorum bacterium or a treated product of said bacterium as an active component, can enhance intestinal barrier function and thereby prevent or treat conditions or diseases associated with intestinal barrier dysfunction. The inventive intestinal barrier function enhancer can be provided in the form of a pharmaceutical composition or a food or beverage.

In other words, as viewed from another aspect, the present invention is directed to a method for preparing a pharmaceutical composition or a food or beverage, the method comprising the step of incorporating an intestinal barrier function enhancer comprising a Lactobacillus crustorum bacterium or a treated product of said bacterium.

Hereunder, the present invention will be more specifically described by way of working examples. However, this invention is not limited to these examples.

### EXAMPLE 1

Evaluation of enhancement of intestinal barrier function by Lactobacillus crustorum bacteria using Caco2 human intestinal epithelial cells

<Materials>

As Lactobacillus crustorum bacteria, there were used Lactobacillus crustorum SAM2695 strain, Lactobacillus crustorum SAM2697 strain, Lactobacillus crustorum SAM2698 strain, and the type culture strain Lactobacillus crustorum JCM 15951^{T}.

The evaluation of enhancement of intestinal barrier function was made using Caco2 human intestinal epithelial cells. This type of cells is derived from human colon cancer and widely used as an in vitro small intestine model. The barrier function and transepithelial electric resistance (TER) value of said cells are known to be highly correlated with each other. A positive change in the TER value in association with coculture of a Lactobacillus crustorum bacterium and Caco2 cells was evaluated as an indicator for enhanced intestinal barrier function.

<Methodology>

### Preparation of Bacterial Strains

At a preculture step, 100 μL each of different bacterial strains was taken from each of their storage vials, seeded on 10 mL of a MRS medium, and cultured at 30° C. for 24 hours. Next, at a main culture step, 10 mL each of the preculture solutions was seeded on 1 L of a MRS medium and cultured at 30° C. for 24 hours. After culture for 24 hours, each of the culture solutions was centrifuged at 8000 rpm for 10 minutes to remove a supernatant. Sterile water was added to wash the culture solutions, which were then centrifuged under the same conditions. After a supernatant was removed, the culture solutions were washed once more and finally suspended in sterile water.

### Preparation of Caco2 Cells

Caco2 cells were seeded on the apical wells of a Millicell 24-well cell culture plate (Merck Millipore) under the condition of 0.7×10⁵ cells/well, and cultured under the condition of 5% CO₂ at 37° C. for two weeks. To prepare a medium, there were used a Dulbecco's modified eagle medium (DMEM) (Sigma-Aldrich, D6429), a fetal bovine serum (FBS) (Equiteck Bio, Inc.), and an antibiotic-antimycotic mixed stock solution (100×) (Nacalai Tesque, Inc.) servicing as an antibiotic. The medium was prepared by adding 50 mL of FBS and 5 mL of the antibiotic to 500 mL of DMEM. The medium was added to each apical well in an amount of 400 μL, and to each basolateral well in an amount of 800 μL. The medium was replaced every two or three days.

Those Caco2 monolayer cells which showed a transepithelial electrical resistance of not less than 300 Ω•cm² before addition of a Lactobacillus crustorum bacterium were regarded as having well-formed tight junctions and used in the assay.

Test Procedure

Forty microliters each of the bacterium suspensions prepared at 1×10⁹ cells/mL (or a medium in the case of the control) was added to the Caco2 cells cultured for two weeks, and cocultured at 37° C. under the condition of 5% CO₂ for two hours. For transepithelial electrical resistance measurements, the cells were measured for their transepithelial electrical resistances before and 24 hours after the addition of a Lactobacillus crustorum bacterium, using a Millicell ERS-2 electronic voltmeter (Merck Millipore). The tests were conducted with n=6 per group.

<Results>

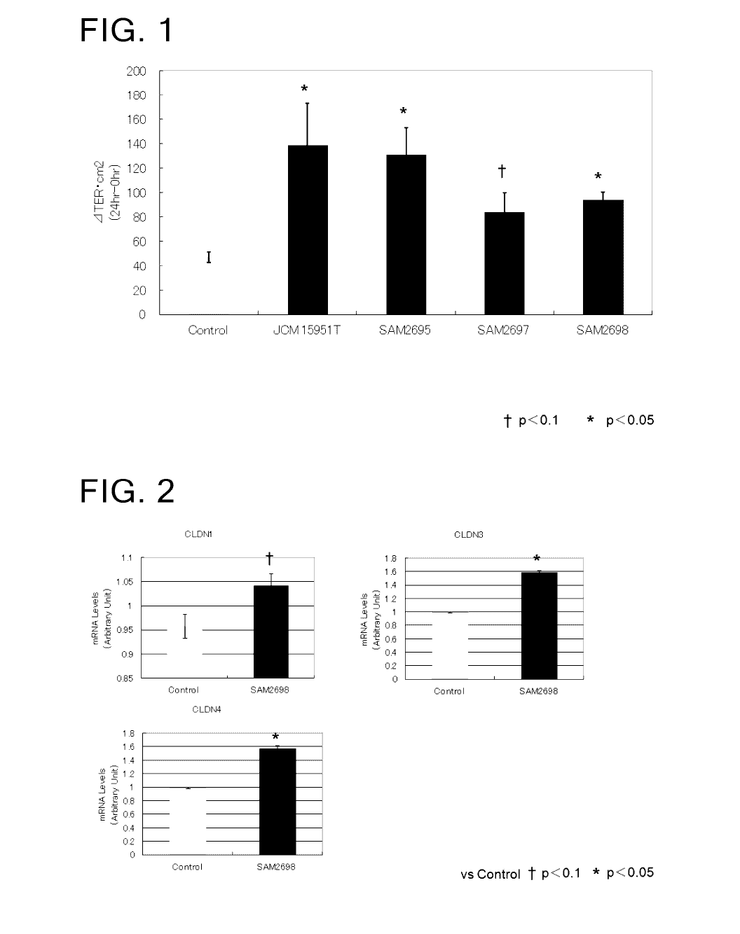

The results are shown in FIG. 1.

The Lactobacillus crustorum JCM 15951^{T}, SAM 2695 and SAM 2698 strains showed a significant increase in TER as compared to the control. The SAM 2697 strain showed an upward trend in TER.

### EXAMPLE 2

Microarray analysis in the Caco2 intestinal epithelial cells cocultured with a Lactobacillus crustorum bacterium

Caco2 cells and Lactobacillus crustorum SAM 2698 strain were cocultured, and examined for the expression of intestinal barrier-related genes.

<Material>

### Preparation of a Bacterium

As a Lactobacillus crustorum bacterium, Lactobacillus crustorum SAM 2698 strain was used.

At a preculture step, 100 μL of the bacterial strain was taken from its storage vial, seeded on 10 mL of a MRS medium, and cultured at 30° C. for 24 hours. Then, 10 mL of the preculture solution was seeded on 1 L of a MRS medium and cultured at 30° C. for 24 hours. Next, the culture solution was centrifuged at 8000 rpm for 10 minutes to remove a supernatant. Sterile water was added to wash the culture solution, which was then centrifuged under the same conditions. After a supernatant was removed, the culture solution was washed once more and finally suspended in sterile water.

Preparation of Caco2 Cells

Caco2 cells were seeded on a 12-well cell culture plate (BD Falcon) under the condition of 0.63×10⁵ cells/2 mL/well, and cultured under the condition of 5% CO₂ at 37° C. for two weeks. To prepare a medium, there were used a Dulbecco's modified eagle medium (DMEM) (Sigma-Aldrich, D6429), a fetal bovine serum (FBS) (Equiteck Bio, Inc.), and an antibiotic-antimycotic mixed stock solution (100×) (Nacalai Tesque, Inc.) servicing as an antibiotic. The medium was prepared by adding 50 mL of FBS and 5 mL of the antibiotic to 500 mL of DMEM. The medium was replaced every two or three days.

<Methodology>

### Test Method

The Lactobacillus crustorum suspension in DMEM was added at a density of 1×10⁹ cells/2 mL/well to the Caco2 cells cultured for two weeks, and cocultured for 5 hours. All the tests were conducted with n=3. After 5 hours, a supernatant was removed, and the precipitate was washed with PBS(−). RNA was prepared from the recovered cells using an RNeasy mini kit (Qiagen).

Microarray Analysis

A request was made to Takara Bio Inc. to conduct an Agilent Expression Array analysis with Human SurePrint G3 (8×60 k) ver 2.0.

Microarray Analysis Results

FIG. 2 shows the results of the analysis focusing on Claudin genes—tight junction—related genes. As compared to the control, the Caco2 cells cocultured with Lactobacillus crustorum SAM 2698 strain showed an increase in the expression of Claudin 1 (CLDN 1) (by about 1.1 time). Those cells also showed a significant increase in the expression of CLDN 3 and CLDN 4 (by about 1.6 times each).

### EXAMPLE 3

Change in the expression of CLDN 1 in small-intestinal epithelial tissue of mice treated with a Lactobacillus crustorum bacterium

Mice were treated with viable Lactobacillus crustorum SAM 2698 strain and examined for change in the expression of CLDN 1 in their small-intestinal epithelial tissue.

<Materials>

As a Lactobacillus crustorum bacterium, Lactobacillus crustorum SAM 2698 strain was used.

The test animals used were C57BL/6J male mice aged 7 weeks. As the groups to be tested, there were included two different groups each treated with sterile water or viable Lactobacillus crustorum SAM 2698 strain. Each group consisted of n=6 mice.

<Methodology>

### Test Procedure

To administer the viable strain, 250 μL of the bacterium suspension at 1.2×10¹⁰ cells/mL was orally applied to the mice in a single dose (at a dose of 3.0×10⁹ cells/mouse). Five hours after the administration, small-intestinal epithelial tissue was collected.

RNA extraction from small-intestinal epithelial tissue

Glass beads with a size of 150-212 μm (Sigma) were added to the collected small-intestinal epithelial tissue, and the tissue was treated at 3000 rpm for 3 minutes using a Multibeads shocker (Yasui Kikai Corporation). Then, RNA was prepared using an RNeasy mini kit (Qiagen) according to the kit's protocol.

Design of Primers

The primer sequences for the murine CLDN 1 gene were designed on the basis of the published gene sequence information. These primer sequences are as follows: murine CLDN 1 forward primer: CCTATGCTGGGGACAACATC; and murine CLDN 1 reverse primer: TGCACCTCATCATCTTCCAG. In this expression analysis, GAPDH gene expression was used as a control. The primer sequences for the GAPDH gene were designed by making reference to the information on Housekeeping Gene Primers for RT-PCR, which is published by ShineGene Molecular Biotech, Inc. These primer sequences are as follows: Mouse GAPDH F49: GGTGAAGGTCGGTGTGAACG; and R281: CTCGCTCCTGGAAGATGGTG.

Realtime PCR Conditions

The expression analysis was performed using realtime PCR (Step One Plus, Applied Biosystems).

The extracted RNA was adjusted to 100 ng/μL with RNase-free water, and 2 μL of the RNA solution was added to 18 μL of the reaction solution containing 10 μM forward and reverse primers to perform realtime PCR. The realtime PCR was performed using the Power SYBR® Green RNA-to-CTTM 1-Step Kit (Applied Biosystems) according to the kit's protocol.

<Results>

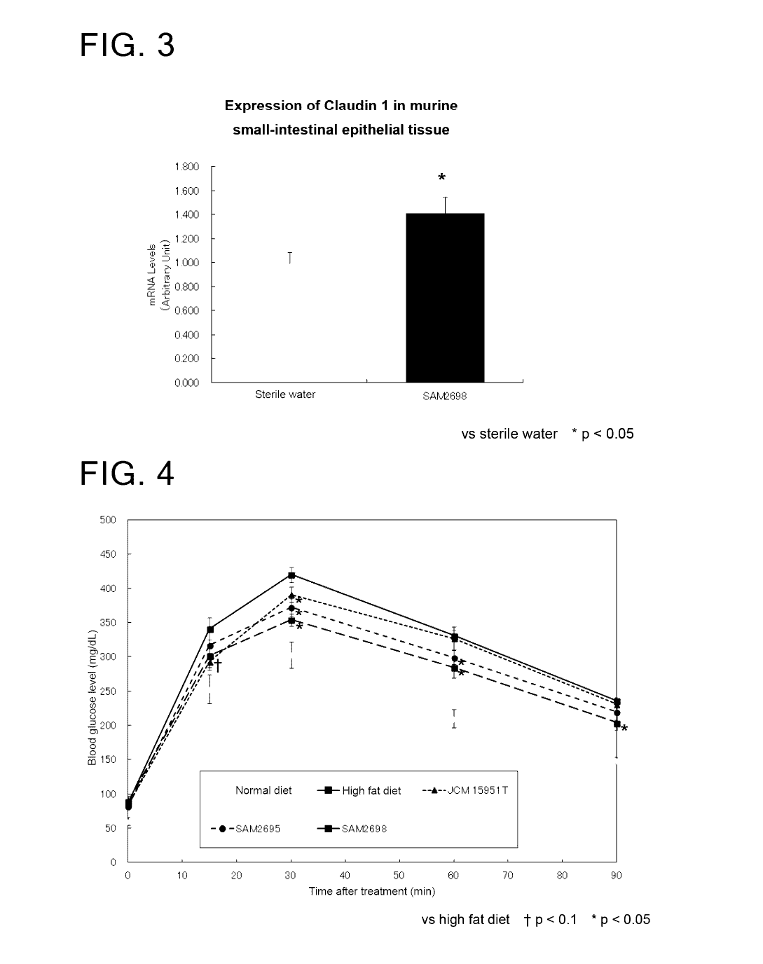

FIG. 3 shows the results of the analysis focusing on the tight junction-related gene CLDN 1. As compared to the group treated with sterile water, the Lactobacillus crustorum SAM 2698-treated mice showed a significant increase in the expression of Claudin 1 (CLDN 1) in their small-intestinal epithelial tissue.

### EXAMPLE 4

Inhibitory activities of Lactobacillus crustorum strains against glucose metabolism disorders in high-fat diet-loaded model mice

<Materials>

As Lactobacillus crustorum bacteria, there were used Lactobacillus crustorum SAM2695 strain, Lactobacillus crustorum SAM2698 strain, and the type culture strain Lactobacillus crustorum JCM 15951^{T}.

The test animals to be used were C57BL/6J made mice aged 7 weeks. As the groups to be tested, there were included several different groups each treated with a normal diet, a 60% high fat diet, or a 60% high fat diet plus each of the viable Lactobacillus crustorum strains. Each group consisted of n=10 mice.

<Methodology>

### Test Procedure

The test was done for 25 days. To administer the viable strains, 250 μL of the bacterium suspension at 1.2×10¹⁰ cells/mL was orally applied to the mice in a single dose (at a dose of 3.0×10⁹ cells/day/mouse).

Diets Used in the Animal Test

The normal diet and 60% high-fat diet used were respectively the purified diets each with a 10 kcal % fat (lard) or a 60 kcal % fat (lard), produced by Research Diets Inc.

Glucose Metabolism Disorder Inhibition Test (Glucose Tolerance Test)

Glucose tolerance test was conducted on day 25. A 20% glucose solution was orally applied to the mice at a rate of 1 g/kg (5 mL/kg), and blood was collected from the caudal vein before and 15, 30, 60 and 90 minutes after the administration. Blood glucose was measured using a Glutest Neo Super (Sanwa Kagaku Kenkyusho Co., Ltd.).

Results of glucose metabolism disorder inhibition test (glucose tolerance test)

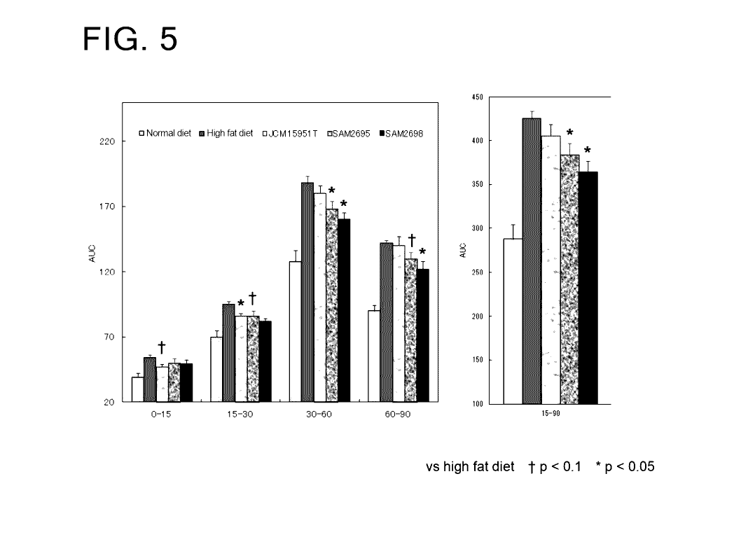

The results are shown in FIGS. 4 and 5.

According to the blood glucose level measurements in the groups after glucose load, as compared to the normal diet treated group, the 60% high-fat diet treated group showed significantly high values for the levels at 15, 30, 60 and 90 minutes after the administration. However, as compared to the 60% high-fat diet treated group, the group treated with a 60% high-fat diet plus Lactobacillus crustorum JCM 15951^{T} showed a trend toward significantly inhibited blood glucose elevation at 15 minutes and a trend toward inhibited blood glucose elevation at 30 minutes. As compared to the 60% high-fat diet treated group, the group treated with a 60% high-fat diet plus Lactobacillus crustorum SAM 2695 showed significant inhibition of blood glucose elevation at 30 and 60 minutes. According to the AUC determined at 15 to 90 minutes, the Lactobacillus crustorum SAM 2695 group showed a significant decrease in blood glucose level as compared to the 60% high-fat diet-treated group. The group treated with a 60% high-fat diet plus Lactobacillus crustorum SAM 2698 strain showed significant inhibition of blood glucose elevation at 30, 60 and 90 minutes. According to the AUC determined at 15 to 90 minutes, the Lactobacillus crustorum SAM 2698 group showed a significant decrease in blood glucose level as compared to the 60% high-fat diet-treated group.

### INDUSTRIAL APPLICABILITY

The intestinal barrier function enhancer of the present invention, which comprises a Lactobacillus crustorum bacterium or a treated product of said bacterium, has a capability of enhancing intestinal barrier function. The inventive intestinal barrier function enhancer can be effectively used for pharmaceutical compositions, foods or beverages which are intended for use in prevention or treatment of various conditions or diseases attributable to intestinal barrier dysfunction, including diabetes, hypertension, hyperlipidemia, obesity, and metabolic syndrome.

1. A food or beverage comprising a Lactobacillus crustorum bacterium or a treated product of said bacterium.

2. The food or beverage according to claim 1, wherein the Lactobacillus crustorum bacterium is Lactobacillus crustorum SAM2695 strain, Lactobacillus crustorum SAM2697 strain, Lactobacillus crustorum SAM2698 strain, Lactobacillus crustorum JCM 15951^{T} strain, or a combination of two or more thereof.

3. The food or beverage according to claim 1, wherein the food or beverage comprises a viable Lactobacillus crustorum bacterium.

4. An intestinal barrier function enhancer comprising a Lactobacillus crustorum bacterium or a treated product of said bacterium.

5. The intestinal barrier function enhancer according to claim 4, wherein the Lactobacillus crustorum bacterium is Lactobacillus crustorum SAM2695 strain, Lactobacillus crustorum SAM2697 strain, Lactobacillus crustorum SAM2698 strain, Lactobacillus crustorum JCM 15951^{T} strain, or a combination of two or more thereof.

6. The intestinal barrier function enhancer according to claim 4, wherein the food or beverage comprises a viable Lactobacillus crustorum bacterium.

7. A pharmaceutical composition for prevention or treatment of at least one condition or disease selected from the group consisting of diabetes, hypertension, hyperlipidemia and obesity, the pharmaceutical composition comprising the intestinal barrier function enhancer according to claim 4.

8. A food or beverage comprising the intestinal barrier function enhancer according to claim 4.

9. The food or beverage according to claim 8, wherein the food or beverage is intended for use in prevention or treatment of at least one condition or disease selected from the group consisting of diabetes, hypertension, hyperlipidemia and obesity.

10. The pharmaceutical composition according to claim 7, wherein the condition or disease is diabetes.

11. The pharmaceutical composition according to claim 7, wherein the condition or disease is a combination of obesity with at least one disease selected from the group consisting of diabetes, hypertension and hyperlipidemia.

12. The pharmaceutical composition, or food or beverage according to claim 7, wherein the diabetes is Type 2 diabetes.

13. A method for preparing a pharmaceutical composition or a food or beverage, the method comprising the step of incorporating the intestinal barrier function enhancer according to claim 4.

14. The method according to claim 13, wherein the pharmaceutical composition or the food or beverage is intended for use in prevention or treatment of at least one condition or disease selected from the group consisting of diabetes, hypertension, hyperlipidemia and obesity.

15. The method according to claim 14, wherein the condition or disease is diabetes.

16. The method according to claim 14, wherein the condition or disease is a combination of obesity with at least one disease selected from diabetes, hypertension and hyperlipidemia.

17. The method according to claim 13, wherein the diabetes is Type 2 diabetes.
